SEQUENCE LISTING (1 sequence)

SEQ ID NO: 1 - protein, 269 aa, Homo sapiens
MERLVIRMPFCHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGYWFSVPLLLSIVSLVILLVLISILLYWKRHRNQDREP

(19) United States
(12) Patent Application Publication
(10) Pub. No.: US 2024/0173266 A1
(43) Pub. Date: May 30, 2024

(54) NANOCARRIERS FOR TARGETING TUMOR ASSOCIATED MACROPHAGES

(71) Applicant: OHIO STATE INNOVATION FOUNDATION, Columbus, OH (US)

(72) Inventors: Natalia Higuita-Castro, Columbus, OH (US); Daniel Gallego-Perez, Columbus, OH (US); Ana Salazar-Puerta, Columbus, OH (US)

(21) Appl. No.: 18/551,746

(22) PCT Filed: Mar. 15, 2022
(86) PCT No.: PCT/US2022/071154

§ 371 Date: Sep. 21, 2023

Related U.S. Application Data
(60) Provisional application No. 63/178,037, filed on Apr. 22, 2021.

(51) Int. Cl. A61K 9/50; C07K 14/705

(52) U.S. Cl. CPC A61K 9/5068; C07K 14/70503; C07K 2319/03

(57) ABSTRACT

Disclosed herein is a nanocarrier system to effectively target TAMs to treat cancer. The disclosed nanocarriers are custom-made extracellular vesicles (EVs) loaded with therapeutic cargo, such as antineoplastic agents, and are functionalized for targeted delivery via decoration with ligands for specific receptors in TAMs. Therefore, also disclosed is a method of inhibiting tumor growth or tumor metastases in a subject in need thereof comprising selectively targeting TAMs.

### CROSS-REFERENCE TO RELATED APPLICATIONS

This application claims benefit of U.S. Provisional Application No. 63/178,037, filed Apr. 22, 2021, which is hereby incorporated herein by reference in its entirety.

### SEQUENCE LISTING

This application contains a sequence listing filed in electronic form as an ASCII.txt file entitled “321501_2550_Sequence_Listing_ST25” created on Mar. 14, 2022, and having 2,625 bytes. The content of the sequence listing is incorporated herein in its entirety.

### BACKGROUND

Tumors harbor dynamic microenvironments in which cancer cells are intimately associated with non-transformed host cells. The tumor-associated stroma is considered to play an important role during tumor growth, influencing phenomena such as angiogenesis, metastasis and immune suppression. As such, the stroma forms an attractive target for diagnostic and therapeutic applications.

Different myeloid cells are important components of the tumor stroma. Myeloid cells are frequently found to infiltrate tumors and have been linked to diverse tumor-promoting activities. In particular, tumor-associated macrophages (TAMs) are an important component of the tumor stroma, both in murine models and human patients. TAMs can promote tumor-growth by affecting angiogenesis, immune suppression and invasion and metastasis.

Macrophages are plastic cells that can adopt different phenotypes depending on the immune context. Microenvironmental stimuli can drive a macrophage either towards a “classical” (M1) or an “alternative” (M2) activation state, two extremes in a spectrum. M1 macrophages are typically characterized by the expression of pro-inflammatory cytokines, inducible nitric oxide synthase 2 (Nos2) and MHC Class II molecules. M2 macrophages have a decreased level of the aforementioned molecules and are identified by their signature-expression of a variety of markers, including arginase-1 and mannose and scavenger receptors. It has been suggested that TAMs display a M2-like phenotype.

### SUMMARY

Disclosed herein is a nanocarrier system to effectively target TAMs to treat cancer. The disclosed nanocarriers are custom-made extracellular vesicles (EVs) loaded with therapeutic cargo, such as antineoplastic agents, and are functionalized for targeted delivery via decoration with ligands for specific receptors in TAMs.

These nanocarriers can be obtained after transfection of cells in vitro or tissues in vivo using diverse transfection techniques (e.g. bulk electroporation, nanoelectroporation, tissue nanotransfection, viral transfection). As shown in FIG. 1, loading EVs with antineoplastic agents, and/or decoration with cell-targeting ligands, can be achieved by transfecting, for example, plasmid DNA encoding for the specific cargo or ligand. The cargo could be varied depending on the inflammatory pathway that needs to be regulated, and the surface decoration can be varied depending on the target cell type.

For example, TAMs can be targeted using plasmid genes that can encode for membrane glycoprotein CD200. EVs functionalized with CD200 preferentially interact with the CD200R receptor in TAMs. Also disclosed are EVs functionalized with CD200R preferentially interact with the CD200 receptor on CD200-expressing cells.

CD200-CD200R interaction is crucial in regulating the tumor microenvironment by affecting tumor-associated myeloid cells (TAMCs), such as tumor associated macrophages, dendritic cells, and myeloid-derived suppressor cells, which express CD200R. CD200 is highly expressed in cancer cells of different human tumors, T cells, and endothelial cells, and inhibits the function of TAMCs, inhibiting tumor formation, metastasis and affecting predisposition to T cell therapy. Therefore, targeting CD200-CD200R interaction can provide an option for immunotherapy.

There are some structural similarities that CD200 shares with checkpoint molecules (PD-1, CTLA4, and CD47, which leads to immune checkpoint blockade). Different studies have shown a downregulation of the anti-tumor immunity associated with the CD200-CD200R pathway in neuroblastoma tumors (reduced amount of CD8+ and CD4+ T cells at the tumor site). Therefore, there is a potential benefit for neuroblastomas when using an anti-CD200 antibody to block this pathway.

Neurons also express CD200, as a self-protection mechanism, which interacts to CD200R on the surface of microglial cells, preventing thus a secondary neuronal injury caused by those cells (e.g., stroke immunotherapy). After stroke, microglia get activated and start producing a lot of proinflammatory cytokines that can damage neurons as well as the blood-brain barrier, which in turn can affect neurogenesis. Damaged caused by microglia can be restored by the receptor-ligand interaction CD200-CD200R, since the binding leads to the inhibition in the production of pro-inflammatory mediators. Another example would be Parkinson's disease.

A model of murine multiple sclerosis is the autoimmune encephalomyelitis (EAE), which is characterized by the activation of peripheral macrophages, T cells, and granulocytes, that migrate to the central nervous system, leading to microglial activation, tissue damage, and neurological deficits (e.g., paralysis). When CD200 is absent, there is an increase of the disease onset and progression.

Human rheumatoid arthritis, an inflammatory autoimmune disease of the joints, can be mimic with a collagen-induced arthritis (CIA) model. Stimulation of CD200R by CD200, delivers inhibitory signals to myeloid cells. Studies have shown that using CD200-Fc (agonist for CD200R) can be used to block the development of collagen-induced arthritis(5, 6).

CD200-CD200R interaction has shown to increased graft acceptance by the suppression of inflammatory responses and the induction of regulatory T cells.

The lung highly express CD200 (e.g., alveolar macrophages, mast cells, and dendritic cells) and it is thought to play a critical role in pulmonary immunoregulation. More specifically, the airway epithelium present high levels of CD200, which interacts to CD200R1 on dendritic cells and alveolar macrophages. In absence of inflammatory stimuli, this interaction inhibits their activation. During asthma, those levels of CD200 are downregulated on the airway epithelium, and lung resident immune cells show over-expression of CD200R.

TAMs can also be targeted using plasmid genes that can encode for CD163, scavenger receptors (SR-A, CD204), Tyro3, Axl, and Mertk (TAM receptor tyrosine kinases), FRβ (ligand: immunotoxin), mannose receptors (MMR, CD206), TIM-3 blocking antibody, VEGF, cMAF, MGL1, or MGL2.

TAMs can be targeted using IL4, IL13, IL10, TGFβ, PGE2 (activate M2), Growth arrest specific factor 6 (Gas6) and Protein S (Pros1), are specific ligands that activate Tyro3, Axl, Mertk, Melittin (MEL, binds preferentially to CD206 in M2 macrophages), PD-1, SIRPa, LILRB1, or Siglec-10 (receptors).

As shown in FIG. 2, surface-decorated EVs can also be loaded with therapeutic cargo that are membrane-permeable pharmacological compounds that can diffuse into the EVs via a concentration gradient.

Therefore, also disclosed herein is a method of inhibiting tumor growth or tumor metastases in a subject in need thereof comprising selectively targeting TAMs. As a specific embodiment, the above method comprises administering to the mammal a pharmaceutically effective amount of the nanocarrier system disclosed herein.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic representation of production of custom-made nanocarriers loaded with anti-inflammatory cargo and/or decorated with cell-targeting ligands after transfection of cells or tissues.

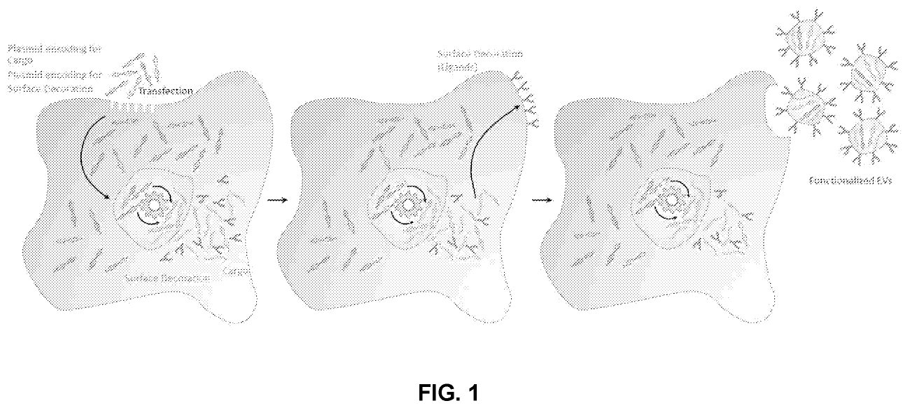

FIG. 2 is a schematic representation of surface-decorated EVs loaded with other anti-inflammatory cargo, for example, a membrane-permeable pharmacological compound.

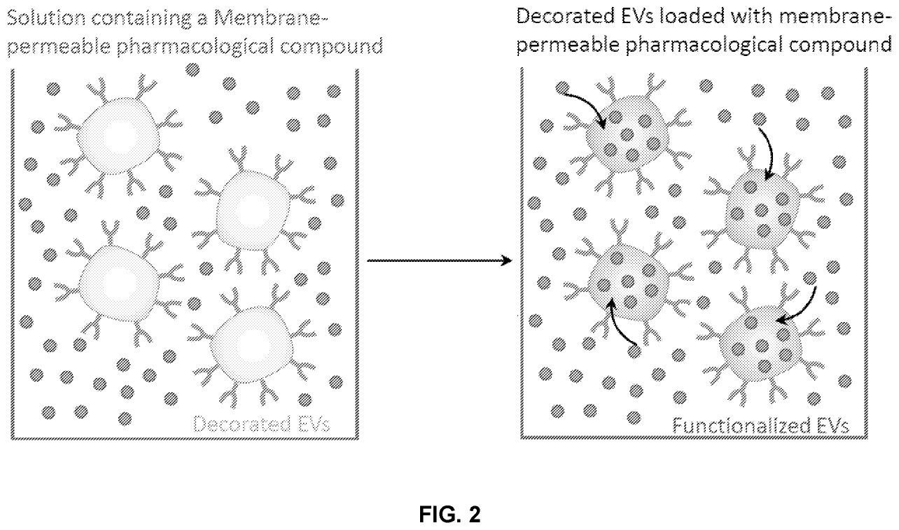

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The disclosed EVs can in some embodiments be any that can be produced by a cell. Cells secrete extracellular vesicles (EVs) with a broad range of diameters and functions, including apoptotic bodies (1-5 μm), microvesicles (100-1000 nm in size), and vesicles of endosomal origin, known as exosomes (50-150 nm).

The disclosed extracellular vesicles may be prepared by methods known in the art. For example, the disclosed extracellular vesicles may be prepared by expressing in a eukaryotic cell an mRNA that encodes the cell-targeting ligand. In some embodiments, the cell also expresses an mRNA that encodes a therapeutic cargo. The mRNA for the cell-targeting ligand and the therapeutic cargo may be expressed from vectors that are transfected into suitable production cells for producing the disclosed EVs. The mRNA for the cell-targeting ligand and the therapeutic cargo may be expressed from the same vector (e.g., where the vector expresses the mRNA for the cell-targeting ligand and the anti-inflammatory cargo from separate promoters), or the mRNA for the cell-targeting ligand and the therapeutic cargo may be expressed from separate vectors. The vector or vectors for expressing the mRNA for the cell-targeting ligand and the therapeutic cargo may be packaged in a kit designed for preparing the disclosed extracellular vesicles.

Also disclosed is a composition comprising an EV containing the disclosed targeting ligands. In some embodiments, the EV is loaded with a therapeutic cargo. Also disclosed is an EV producing cell engineered to secrete the disclosed EVs.

EVs, such as exosomes, are produced by many different types of cells including immune cells such as B lymphocytes, T lymphocytes, dendritic cells (DCs) and most cells. EVs are also produced, for example, by glioma cells, platelets, reticulocytes, neurons, intestinal epithelial cells and tumor cells. EVs for use in the disclosed compositions and methods can be derived from any suitable cell, including the cells identified above. Non-limiting examples of suitable EV producing cells for mass production include dendritic cells (e.g., immature dendritic cell), Human Embryonic Kidney 293 (HEK) cells, 293T cells, Chinese hamster ovary (CHO) cells, and human ESC-derived mesenchymal stem cells. EVs can also be obtained from autologous patient-derived, heterologous haplotype-matched or heterologous stem cells so to reduce or avoid the generation of an immune response in a patient to whom the exosomes are delivered. Any EV-producing cell can be used for this purpose.

Also disclosed is a method for making the disclosed EVs loaded with therapeutic cargo that involves culturing the disclosed EV-producing cell engineered to secrete the disclosed EVs. The method can further involve purifying EVs from the cells.

EVs produced from cells can be collected from the culture medium by any suitable method. Typically, a preparation of EVs can be prepared from cell culture or tissue supernatant by centrifugation, filtration or combinations of these methods. For example, EVs can be prepared by differential centrifugation, that is low speed (<20000 g) centrifugation to pellet larger particles followed by high speed (>100000 g) centrifugation to pellet EVs, size filtration with appropriate filters, gradient ultracentrifugation (for example, with sucrose gradient) or a combination of these methods.

The disclosed EVs can be targeted to TAMs by expressing on the surface of the EVs a targeting moiety which binds to a cell surface moiety expressed on the surface of the cell. Examples of suitable targeting moieties are short peptides, scFv and complete proteins, so long as the targeting moiety can be expressed on the surface of the exosome. Peptide targeting moieties may typically be less than 100 amino acids in length, for example less than 50 amino acids in length, less than 30 amino acids in length, to a minimum length of 10, 5 or 3 amino acids.

In some embodiments, TAMs can be targeted using membrane glycoprotein CD200 or CD200R.

EVs functionalized with CD200 preferentially interact with the CD200R receptor in TAMs. Cells that express CD200 on their surface include Thymocytes, B cells, T cells, Follicular dendritic cells, Tonsil follicles, Kidney glomeruli, Syncytiotrophoblasts, Endothelial cells (CNS), Neurons, Smooth muscle cells, Epithelial cells, Oligodendrocytes, Astrocytes, and Human cancer cells.

Cells that express CD200R on their surface include Microglia, Myeloid cells (macrophages, neutrophils, and mast cells), some subsets of T cells (regulatory T cells [Tregs]), and lower expression in dendritic cells (DCs).

In some embodiments, the targeting moiety is CD200 having the amino acid sequence: MERLVIRMPFCHLSTYSLVWVMAAVVLCTAQVQVVTQDEREQLYTPASLKCSLQNAQE ALIVTWQKKKAVSPENMVTFSENHGVVIQPAYKDKINITQLGLQNSTITFWNITLEDEGC YMCLFNTFGFGKISGTACLTVYVQPIVSLHYKFSEDHLNITCSATARPAPMVFWKVPRS GIENSTVTLSHPNGTTSVTSILHIKDPKNQVGKEVICQVLHLGTVTDFKQTVNKGYWFSV PLLLSIVSLVILLVLISILLYWKRHRNQDREP (SEQ ID NO:1), or a variant and/or fragment thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 1 that can interact with lung macrophages.

In some embodiments, the targeting ligand is a fragment of CD200 comprising at least 100, 110, 120, 130, 140, 141, 142, 143, 144, 145, 156, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, or 269 contiguous amino acids of SEQ ID NO:1 or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to this fragment that can interact with lung macrophages.

The cell targeting ligand can in some cases be expressed on the surface of the EV by expressing it as a fusion protein with an exosomal or lysosomal transmembrane protein. A number of proteins are known to be associated with exosomes; that is they are incorporated into the exosome as it is formed. Examples include but are not limited to Lamp-1, Lamp-2, CD13, CD86, Flotillin, Syntaxin-3, CD2, CD36, CD40, CD40L, CD41a, CD44, CD45, ICAM-1, Integrin alpha4, LiCAM, LFA-1, Mac-1 alpha and beta, Vti-IA and B, CD3 epsilon and zeta, CD9, CD18, CD37, CD53, CD63, CD81, CD82, CXCR4, FcR, GluR2/3, HLA-DM (MHC II), immunoglobulins, MHC-I or MHC-II components, TCR beta and tetraspanins.

The disclosed extracellular vesicles further may be loaded therapeutic cargo, where the extracellular vesicles deliver the agent to a target cell. Suitable therapeutic cargo include but are not limited to therapeutic drugs (e.g., small molecule drugs), therapeutic proteins, and therapeutic nucleic acids (e.g., therapeutic RNA). In some embodiments, the disclosed extracellular vesicles comprise a therapeutic RNA (also referred to herein as a “cargo RNA”). For example, in some embodiments the fusion protein containing the cell-targeting motif also includes an RNA-domain (e.g., at a cytosolic C-terminus of the fusion protein) that binds to one or more RNA-motifs present in the cargo RNA in order to package the cargo RNA into the extracellular vesicle, prior to the extracellular vesicles being secreted from a cell. As such, the fusion protein may function as both of a “cell-targeting protein” and a “packaging protein.” In some embodiments, the packaging protein may be referred to as extracellular vesicle-loading protein or “EV-loading protein.”

In some embodiments, the cargo RNA is a miRNA, shRNA, mRNA, ncRNA, sgRNA or any combination thereof. The cargo RNA of the disclosed extracellular vesicles may be of any suitable length. For example, in some embodiments the cargo RNA may have a nucleotide length of at least about 10 nt, 20 nt, 30 nt, 40 nt, 50 nt, 100 nt, 200 nt, 500 nt, 1000 nt, 2000 nt, 5000 nt, or longer. In other embodiments, the cargo RNA may have a nucleotide length of no more than about 5000 nt, 2000 nt, 1000 nt, 500 nt, 200 nt, 100 nt, 50 nt, 40 nt, 30 nt, 20 nt, or 10 nt. In even further embodiments, the cargo RNA may have a nucleotide length within a range of these contemplated nucleotide lengths, for example, a nucleotide length between a range of about 10 nt-5000 nt, or other ranges. The cargo RNA of the disclosed extracellular vesicles may be relatively long, for example, where the cargo RNA comprises an mRNA or another relatively long RNA.

In some embodiments, the therapeutic cargo is a membrane-permeable pharmacological compound that is loaded into the EV after it is secreted by the cell. The disclosed method can be applied to load EVs with clinically relevant lipophilic compounds and other membrane permeable compounds. In some embodiments, the therapeutic cargo is loaded into the EVs by diffusion via a concentration gradient.

In some embodiments, the therapeutic cargo is an antineoplastic agent. In some embodiments, the therapeutic cargo is Trabectedin, Lurbinectedin (TAM depletion), Pexidartinib, Paclitaxel, Docetaxel Zoledronic acid (repolarization toward M1), Doxorubicin and Lapatinib, IL-6, IL-12, IL-23, TNFα (pro-inflammatory mediators), anti-CD47 (e.g., Hu5F9-G4 mAb, CC-90002 mAb, SRF231 mAb, TTI-62), anti-SIRPα (e.g., MY-1), anti-CSF-1R (e.g., RG7155, FPA008, BLZ945, ARRY-382, JNJ-40346527, PLX3397), anti-CSF1 (e.g., MCS110), PD-1/PD-L1 inhibitors, anti-CD40 (e.g., CP-870,893, APX005M, RO7009789), PI3K inhibitors (e.g., IPI549), or anti-CD204 (e.g., immunotoxin).

Also contemplated herein are methods for using the disclosed EVs. For example, the disclosed extracellular vesicles may be used for delivering the disclosed therapeutic cargo to a target TAM, where the methods include contacting the target TAM with the disclosed EVs. Therefore, also disclosed herein is a method of treating a cancer in a subject, that involves administering to the subject a therapeutically effective amount of a composition containing cargo-loaded lung-targeted EVs disclosed herein. In some embodiments, the cancer is Ovarian carcinoma, Lung carcinoma, Breast cancer, melanoma, Leukemia, sarcoma, neurofibroma, acute myeloid leukemia, prostate cancer, pancreatic cancer, bone metastases, glioblastoma, kidney cancer, neuroblastoma, and colorectal cancer.

The disclosed EVs may be formulated as part of a pharmaceutical composition for treating tumors and the pharmaceutical composition may be administered to a patient in need thereof to deliver the cargo to target tumors.

The disclosed EVs may be administered to a subject by any suitable means. Administration to a human or animal subject may be selected from parenteral, intramuscular, intracerebral, intravascular, subcutaneous, or transdermal administration. Typically the method of delivery is by injection. Preferably the injection is intramuscular or intravascular (e.g. intravenous). A physician will be able to determine the required route of administration for each particular patient.

The EVs are preferably delivered as a composition. The composition may be formulated for parenteral, intramuscular, intracerebral, intravascular (including intravenous), subcutaneous, or transdermal administration. Compositions for parenteral administration may include sterile aqueous solutions which may also contain buffers, diluents and other suitable additives. The EVs may be formulated in a pharmaceutical composition, which may include pharmaceutically acceptable carriers, thickeners, diluents, buffers, preservatives, and other pharmaceutically acceptable carriers or excipients and the like in addition to the EVs.

Parenteral administration is generally characterized by injection, such as subcutaneously, intramuscularly, or intravenously. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof. Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include sodium chloride injection, ringers injection, isotonic dextrose injection, sterile water injection, dextrose and lactated ringers injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone.

Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment. The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations can be packaged in an ampoule, a vial or a syringe with a needle. All preparations for parenteral administration should be sterile, as is known and practiced in the art.

A therapeutically effective amount of composition is administered. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual constructs, and can generally be estimated based on EC50s found to be effective in vitro and in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific construct, the age, weight and condition of the subject to be treated, the severity of the disease and the frequency and route of administration. Different dosages of the construct may be administered depending on whether administration is by intramuscular injection or systemic (intravenous or subcutaneous) injection.

Preferably, the dose of a single intramuscular injection is in the range of about 5 to 20 μg. Preferably, the dose of single or multiple systemic injections is in the range of 10 to 100 mg/kg of body weight.

Due to construct clearance (and breakdown of any targeted molecule), the patient may have to be treated repeatedly, for example once or more daily, weekly, monthly or yearly. Persons of ordinary skill in the art can easily estimate repetition rates for dosing based on measured residence times and concentrations of the construct in bodily fluids or tissues. Following successful treatment, it may be desirable to have the patient undergo maintenance therapy, wherein the construct is administered in maintenance doses, ranging from 0.01 mg/kg to 100 mg per kg of body weight, once or more daily, to once every 20 years.

A number of embodiments of the invention have been described. Nevertheless, it will be understood that various modifications may be made without departing from the spirit and scope of the invention. Accordingly, other embodiments are within the scope of the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

What is claimed is:

1. A method of treating cancer in a subject, comprising administering to the subject a therapeutically effective amount of an extracellular vesicle decorated with CD200R targeting ligands.

2. The method of claim 1, wherein the extracellular vesicle comprises a fusion protein comprising CD200 and an exosomal or lysosomal transmembrane protein.

3. The method of claim 2, wherein the CD200 comprises the amino acid sequence SEQ ID NO:1, or a fragment of CD200 comprising at least 250 contiguous amino acids of SEQ ID NO:1, or a variant thereof having at least 95% identity to SEQ ID NO:1 or the fragment thereof that can interact with lung macrophages.

4. The method of claim 1, wherein the extracellular vesicle further comprises a therapeutic cargo.

5. The method of claim 4, wherein the therapeutic cargo is an anti-neoplastic agent.
